(19) United States
(12) Patent Application Publication
(10) Pub. No.: US 2025/0307887 A1
(43) Pub. Date: Oct. 2, 2025

(54) SURGERY EVIDENCE REPORT GENERATION

(71) Applicant: Kaliber Labs Inc., San Francisco, CA (US)

(72) Inventors: Chandra JONELAGADDA, San Francisco, CA (US); Aneesh JONELAGADDA, San Francisco, CA (US); Bipul KUMAR, San Francisco, CA (US); Abhishek DEY, San Francisco, CA (US); Helal Uddin MULLAH, San Francisco, CA (US); Sarfaraz JELIL, San Francisco, CA (US)

(21) Appl. No.: 18/864,893

(22) PCT Filed: May 11, 2023
(86) PCT No.: PCT/US2023/066876

§ 371 Date: Nov. 11, 2024

Related U.S. Application Data
(60) Provisional application No. 63/340,921, filed on May 11, 2022.

(51) Int. Cl. G06Q 30/04; G06V 10/82; G06V 20/40; G06V 20/52; G06V 20/60; G06V 40/10; G16H 20/40; G16H 40/20; G16H 70/20; G16H 70/60

(52) U.S. Cl. CPC G06Q 30/04; G06V 10/82; G06V 20/46; G06V 20/52; G06V 20/60; G06V 40/10; G16H 20/40; G16H 40/20; G16H 70/20; G16H 70/60; G06V 2201/034

(57) ABSTRACT

A system and method are disclosed for generating a surgery evidence report and/or a billing report that may be used to provide verification and evidence of one or more performed surgical procedures. The systems and methods may receive a surgical video and apply one or more neural networks to identify and detect elements or activities within the surgical video to support (provide evidence) the performance of surgical procedures.

### CLAIM OF PRIORITY

This patent application claims priority to U.S. provisional patent application No. 63/340,921, titled “SURGERY EVIDENCE REPORT GENERATION”, filed on May 11, 2022, herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The present embodiments relate generally to surgery and more specifically to generating a surgery report associated with a particular surgery.

### BACKGROUND

A surgeon's operative notes, dictated after the surgery, are the ground truth on which the surgery centers and the surgeon's practice bill the insurers. The surgery centers get reimbursed for the implants and for the use of the facility, whereas the surgeon's practice gets reimbursed for the procedures and the surgical activity performed. Surgeons spend a considerable amount of time documenting the surgery and describing the complexity of related surgical procedures. In some cases, intraoperative findings could differ from preoperative diagnostics which may form the basis of the surgical authorization obtained from the patient's insurers

When the preauthorization differs from the intraoperative findings, the surgeons proceed with procedures as required. Insurers typically flag the discrepancies between preauthorization and performed procedures and demand evidence from the practice to justify the medical necessity for the procedure that was performed. The practice has to furnish evidence, based on the operative notes. In some cases, the dispute between the practice and the insurers may take several weeks to complete. Any perceived lack of objective evidence for the medical necessity and for the performance of the performed procedures may considerably complicate the process.

Thus, it would be helpful to provide methods and apparatuses for generating and/or validating surgical reports.

### SUMMARY OF THE DISCLOSURE

Described herein are apparatuses, systems, and methods to generate a surgical report, including an initial surgical report. The surgical report may be generated based on one or more neural networks. The surgical report may describe detected surgical procedures. The surgical report may be reviewed and corrected by the patient's surgeon. These corrections may be used to generate a billing report that may be used to generate a bill for a surgical operation. In general, these surgical reports may be referred to as initial surgical reports, although they may be reviewed and/or subsequently finalized and//or modified.

Any of the methods and apparatuses (e.g., systems, including software) described herein may be used to generate a surgical report describing detected billable activities. Any of the methods may include receiving a surgical video of a surgical procedure performed on a patient, identifying one or more surgical tools in the surgical video, detecting surgical activity within the surgical video, and determining one or more billable activities based on the identified surgical tools and the detected surgical activities.

Any of the methods described herein may also include recognizing a patient's anatomy in the surgical video, where determining the one or more billable activities is based, at least in part, on the recognized patient's anatomy. In some examples, recognizing the patient's anatomy may include executing a neural network trained to recognize anatomy in a surgical video.

In any of the methods described herein, identifying the one or more surgical tools includes executing a neural network trained to identify surgical tools in a surgical video. Any of the methods may further include recognizing a pathology in a surgical video, where determining the one or more billable activities is based, at least in part, on the recognized pathology. In some examples, recognizing the pathology may include executing a neural network trained to recognize pathology in a surgical video.

In any of the methods described herein, the billable activities may include a video clip of the detected surgical activity. Still, in any of the methods, the billable activities may include a descriptive text based at least in part on the detected surgical activity.

Any of the methods described herein may include generating an initial surgical report based at least in part on the one or more determined billable activities. In any of the methods, the surgical video may be captured with an orthoscopic camera.

In any of the methods described herein, detecting surgical activity may include executing a neural network trained to detect surgical activity in a surgical video.

Any of the systems described herein may include one or more processors and a memory configured to store instructions that, when executed by the one or more processors, cause the system to receive a surgical video of a surgical procedure performed on a patient, identify one or more surgical tools in the surgical video, detect surgical activity within the surgical video, and determine one or more billable activities based on the identified surgical tools and the detected surgical activities.

Any of the methods described herein may provide an initial surgical report describing detected billable activities. The methods may include determining a plurality of video clips from a surgical video of a surgical procedure performed on a patient, determining a plurality of recommended key frames from the plurality of video clips, and determining one or more billable activities based on the plurality of key frames.

Any of the methods may further include detecting a plurality of surgical phases from the plurality of video clips, where the key frames are based, at least in part, on the plurality of surgical phases. In some examples, any of the methods may further include recognizing one or more stages within at least one of the plurality of surgical phases, where the key frames are based, at least in part, on the one or more stages.

In any of the methods, the key frames may include diagnostic key frames, site preparation key frames, suture passing key frames, anchor insertion key frames, post treatment key frames, or a combination thereof. Furthermore, in any of the methods described herein may further include generating an initial surgical report based at least in part on the key frames.

In any of the methods described herein may include recognizing patient anatomy in one or more of the key frames, where the billable activities are based, at least in part, on the recognized patient anatomy. In some examples, recognizing patient anatomy may include executing a neural network trained to recognize patient anatomy.

In any of the methods described herein may include recognizing a pathology in one or more of the key frames, where the billable activities are based, at least in part, on the recognized pathology. In some examples, recognizing the pathology may include executing a neural network trained to recognize patient pathology.

Any of the methods described herein may include recognizing a surgical tool in one or more of the key frames, where the billable activities are based, at least in part, on the recognized surgical tool. In some examples, recognizing the surgical tool may include executing a neural network trained to recognize one or more surgical tools.

Any of the systems described herein may include one or more processors and a memory configured to store instructions that, when executed by the one or more processors, cause the system to determine a plurality of video clips from a surgical video of a surgical procedure performed on a patient, determine a plurality of recommended key frames from the plurality of video clips, and determine one or more billable activities based on the plurality of key frames.

Any of the methods described herein may include receiving video clips of an operation performed on a patient, determining any modifications to billable activities based, at least in part, on the video clips, and generating a billing report based, at least in part, on the determined modifications.

In any of the methods described herein, determining any modifications to billable activities may include verifying that at least one of the video clips include a particular billable surgical procedure. In any of the methods, determining any modifications to billable activities may include verifying that at least one of the video clips include a particular surgical tool, patient anatomy, or pathology.

In any of the methods described herein, verifying may include executing a neural network trained to recognize surgical tools, patient anatomy, or pathology. In any of the methods, generating the billing report may include mapping detected surgical activity to billable procedures.

Any of the systems described herein may include one or more processors, and a memory configured to store instructions that, when executed by the one or more processors, cause the system to receive video clips of an operation performed on a patient, determine any modifications to billable activities based, at least in part, on the video clips, and generate a billing report based, at least in part, on the determined modifications.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 is a flowchart showing an example method for preparing an initial surgery evidence report.

FIG. 2 shows an example schematic block diagram for generating an initial surgical report.

FIG. 3 shows another example schematic block diagram for generating an initial surgical report.

FIG. 4 is a flowchart depicting an example method for modifying an initial surgical report.

FIG. 5 shows an example schematic block diagram for generating a billing report.

FIG. 6 shows a block diagram of a device that may be one example of any device, system, or apparatus that may provide any of the functionality described herein.

### DETAILED DESCRIPTION

Described herein are systems and methods for generating a surgery report based on a video of a surgical procedure (“surgical video”). The surgical video may be analyzed using one or more neural networks (e.g., artificial intelligence) that have been trained to identify patient anatomy, surgical tools, and/or patient pathologies. Surgical activities may be detected and an associated surgery report may be provided to the surgeon and/or to a patient's file and/or to an insurance carrier.

The surgery report may be reviewed by the surgeon. During this surgery report review, the surgeon may add notes pertaining to the patient which are may not visually perceptible. For example, the surgeon may comment on tissue quality, the feel of the cartilage, and any other aspects that may not be clear or easily determined from the surgical video. The review component lets the surgeon add any such annotations to the surgery report. Annotations may be made in any appropriate manner, including text, graphical and/or images and/or verbal, etc. The resulting report may be referred to herein as an annotated surgery report.

The surgical video may be analyzed in view of the annotated surgery report. In some cases, the surgical video and the annotated surgery report may be reviewed by one or more neural networks that have been trained to identify annotated procedures within the surgical video. The neural networks may be used to find evidence to support the identified annotated procedures and prepare a surgery report that includes support for the identified annotated procedures. This method or apparatus may be used to generate a validated surgical report and/or a billing surgical report. For example, the billing surgical report may be a validated surgical report. In some examples the surgeon may again review the surgical report, further modifying the annotations and the validating review until the surgeon indicates that the report is final, resulting in the final validated and/or billing report.

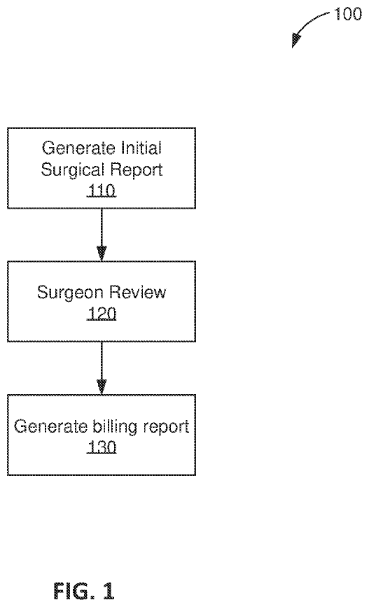

FIG. 1 is a flowchart showing an example method 100 for preparing an initial surgical report. Some examples may perform the operations described herein with additional operations, fewer operations, operations in a different order, operations in parallel, and some operations differently.

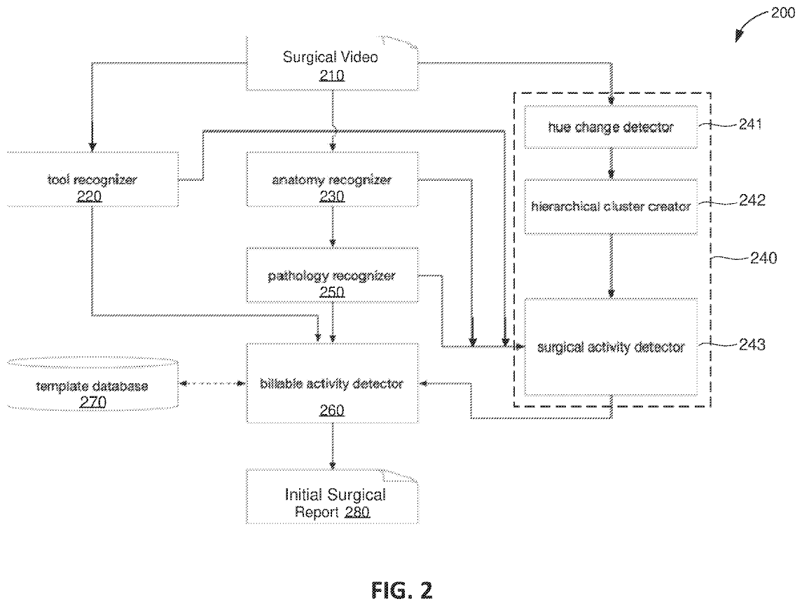

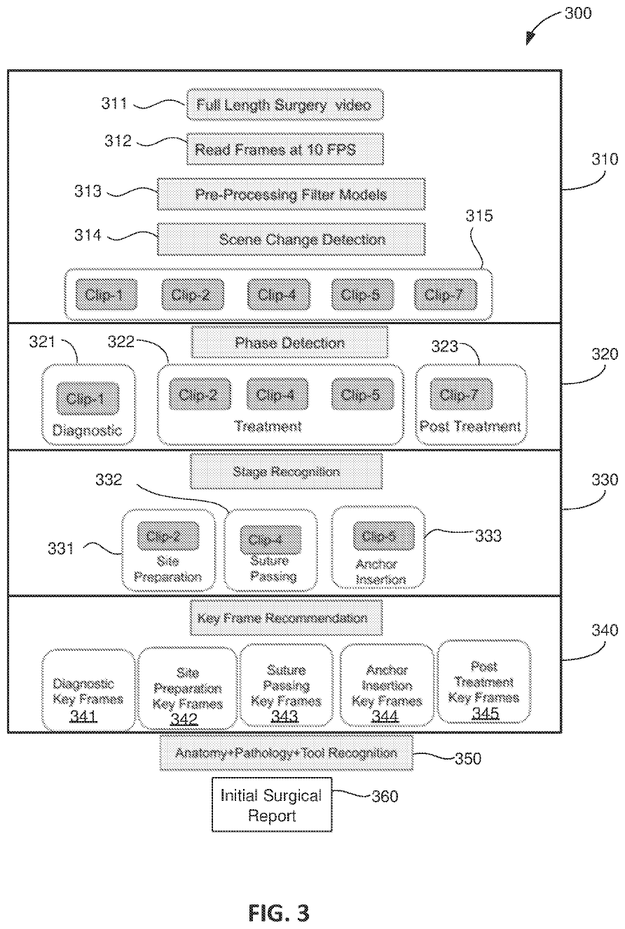

In FIG. 1, the method 100 may include generating an initial surgical report 110. In some examples, the initial surgical report may be generated by analysis of a surgical video by one or more neural networks. The one or more neural networks may identify surgical tools, patient anatomy and pathologies and detect surgical activity from the surgical video that, in turn, are used to generate the initial surgical report. Generation of the initial surgical report is described in more detail below in conjunction with FIGS. 2 and 3.

Next, the surgeon may review the initial surgical report 120. In some examples, during the surgeon's review, the surgeon may annotate the initial surgical report to note additional procedures that may have been performed on the patient, but not included in the initial surgical report.

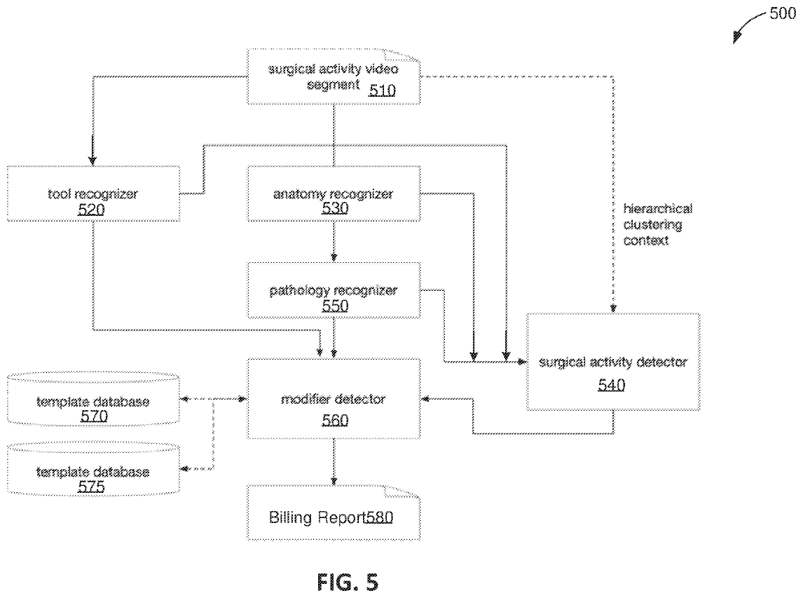

Next, a billing report is generated 130. The billing report may be based on the surgical video, the initial surgical report, and the annotations from the surgeon. The billing report is described in more detail below in reference to FIG. 5.

FIG. 2 shows an example schematic block diagram 200 for generating an initial surgical report 280. The schematic block diagram 200 describes a relationship between various processing steps and surgical data that may be used to generate the initial surgical report 280. In some examples, the processing steps may include executing neural networks with one or more processors (not shown). Thus, the executed neural networks may enable an artificial intelligence-based approach to determining the initial surgical report 280.

First, a surgical video 210 is obtained. The surgical video 210 may have been captured with one or more cameras that were fixed on an operating area. For example, the surgical video 210 may include video clips captured from any number of orthoscopic cameras that may have been focused on, and captured video images from any feasible operating area.

Next, the surgical video 210 is provided to a tool recognizer module 220. In some examples, the tool recognizer module 220 may include one or more neural networks that have been trained to recognize surgical instruments used within the surgical video 210. Noting the use of a particular surgical tool may indicate or corroborate that a particular surgery was performed by the surgeon using the surgical tool.

The surgical video 210 may also be provided to an anatomy recognizer module 230. The anatomy recognizer module 230 may include one or more neural networks that have been trained to recognize a patient's anatomy from the surgical video 210. Knowledge of the patient's anatomy may be useful in determining what surgical procedures may have been performed by the surgeon.

In some examples, information from the anatomy recognizer module 230 may be provided to a pathology recognizer module 250. The pathology recognizer module 250 may include one or more neural networks that may have been trained to recognize and/or identify patient pathologies and/or interoperative findings based on information from the anatomy recognizer module 230.

The surgical video 210 may be provided to video processor module 240. In some examples, the video processor module 240 may modify or detect video color in a hue change detection module 241 and generate or detect hierarchy in a hierarchical cluster creator module 242. Information from the hierarchical cluster creator module 242 may be provided to a surgical activity detector module 243.

The surgical activity detector module 243 may also receive information from the tool recognizer module 220, the anatomy recognizer module 230, and the pathology recognizer module 250. The surgical activity detector module 243 may include one or more neural networks that have been trained to determine and/or detect surgical activity directly and indirectly from the surgical video 210. In some examples, the surgical activity detector module 243 can analyze data from the tool recognizer module 220, the anatomy recognizer module 230, and the pathology recognizer module 250 to identify and/or detect surgical activity. In some variations, the surgical activity detector module 243 determine timestamps of video clips in the surgical video 210 that correspond to any detected surgical activity. In some variations, the surgical activity detector module 243 may also detect anchors, implants, sutures, and the like that may be used during the patient's surgery.

A billable activity detector module 260 may receive data from the tool recognizer module 220, the pathology recognizer module 250, and the surgical activity detector module 243. For example, using recognized tool information (from the tool recognizer module 220), identified pathologies and interoperative findings (from the pathology recognizer module 250), and video timestamps (from the surgical activity detector module 243), the billable activity detector module 260 may determine billable activities.

In some examples, the billable activity detector module 260 may retrieve report billable templates from a template database 270. In some cases, different billable templates may be retrieved for each detected surgical activity. The billable templates may filled-out or populated with data from the tool recognizer module 220, the anatomy recognizer module 230, and the pathology recognizer module 250. Using the billable templates, the billable activity detector module 260 may generate an initial surgical report 280 for review by the surgeon. The initial surgical report 280 may include video clips taken from the surgical video 210 that may correspond to any tools identified by the tool recognizer module 220, any anatomies identified by the anatomy recognizer module 230, any pathologies identified by the pathology recognizer module 250, and/or any surgical activity detected by the surgical activity detector module 243. In some examples, the initial surgical report 280 may include structured labels that identify any feasible detected surgical activity, identified tools, and identified anatomical structures.

The initial surgical report 280 may also include text descriptions, in some cases based on template information from the template database 270, that may be associated with detected billable activities. For example, if the surgical activity detector module 243 detects a video clip that contains evidence of a surgical activity called debridement, the artificial intelligence network (e.g., the tool recognizer module 220, the anatomy recognizer module 230, and the pathology recognizer module 250) can examine the video clip associated with the debridement and list the tools and anatomical structures seen in the video clip. If the list of tools and structures and the underlying activity satisfy the template, then the billable activity is deemed to have taken place.

FIG. 3 shows another example schematic block diagram 300 for generating an initial surgical report 360. In some cases, the processing steps and procedures of the block diagram 300 may be performed additionally or in addition to the processing steps and procedures of the block diagram 200. Thus, the initial surgical report 360 may be another example of the initial surgical report 280 of FIG. 2.

The process may begin with a video processing module 310. The video processing module 310 may receive a surgical video 311 (which may be an example of the surgical video 210 of FIG. 2) and determine a number of video clips 315 from the surgical video 311. An example surgical video 311 may be a full length surgery video. In some examples, the video processing module 310 may process the surgical video 311 by reading data of the surgical video 311 at ten frames per second (FPS) 312. In some cases, reading or processing the surgical video 311 at ten FPS may advantageously simplify video processing by reducing the rate that data (for example, video data) is processed.

The video processing module 310 may pre-process one or more filter models 313. In some examples, the filter models may highlight or make more prominent scene changes. Next, the video processing module 310 may detect scene changes 314. In some examples, the video processing module 310 may include on or more neural networks that may be trained to recognize scene changes. As a result, the video processing module 310 may determine the video clips 315 from the surgical video 311. As an example, FIG. 3 shows that five video clips included in the video clips 315 may be determined from the surgical video 311. In other examples, fewer than five or more than six video clips may be determined.

A phase detection module 320 may receive the video clips 315 from the video processing module 310 and determine one or more surgical phases. In some examples, the phase detection module 320 may include one or more neural networks which may have been trained to determine different surgical phases from a plurality of video clips 315. For example, the neural networks may determine which video clips 315 may be associated with diagnostic 321, treatment 322, and/or post treatment 323 phases. Although three surgical phases are described, in some variations, the phase detection module 320 may determine any feasible number of surgical phases. In the example of FIG. 3, the phase detection module 320 may determine that clip-1 is associated with the diagnostic phase, clip-2, clip-4, and clip-5 are associated with the treatment phase, and clip-7 is associated with the post treatment phase.

A stage recognition module 330 may further examine treatment phases (for example, the treatment phases 322 determined by the phase detection module 320). In some examples, the stage recognition module 330 may include one or more neural networks trained to recognize site preparation stage 331, suture passing stage 332, and/or anchor insertion stage 333 stage, and their related video clips within the treatment phase 322. The site preparation stage 331 include video clips showing surgery site preparation. The suture passing stage 332 may include video clips showing sutures being applied. The anchor insertion stage 333 may include video clips showing anchors being inserted into the patient. In some variations, the stage recognition module 330 may include neural networks trained to recognize site preparation, suture passing, and/or anchor insertion video clips within the treatment phase 322 video clips.

A key frame recommendation module 340 may provide one or more recommended video frames that may be used to generate the initial surgical report 360. For example, based on the video processing module 310, the phase detection module 320, and the stage recognition module 330, the key frame recommendation module 340 may determine or recommend key frames from the surgical video 311. In some examples, the key frame recommendation module 340 may determine diagnostic key frames 341, site preparation key frames 342, suture passing key frames 343, anchor insertion key frames 344, and/or post treatment key frames 345 based on data from the phase detection module 320 and the stage recognition module 330. In some variations, the key frame recommendation module 340 may include one or more neural networks that may be trained to determine the diagnostic key frames 341, the site preparation key frames 342, the suture passing key frames 343, the anchor insertion key frames 344, and/or the post treatment key frames 345 based on data from the phase detection module 320 and the stage recognition module 330. In some variations, any of the key frames may include timestamps of the surgical video 311 and/or an associated example video frames that may illustrate each of the diagnostic key frames 341, the site preparation key frames 342, the suture passing key frames 343, the anchor insertion key frames 344, and/or the post treatment key frames 345.

The diagnostic key frames 341, the site preparation key frames 342, the suture passing key frames 343, the anchor insertion key frames 344, and/or the post treatment key frames 345 are processed with an anatomy recognition, pathology recognition, and tool recognition modules 350. The anatomy recognition, pathology recognition, and tool recognition modules 350 may include one or more neural networks that may have been trained to recognize anatomies, pathologies, and surgical tools, implants, sutures and the like that may be included in the key frames from the key frame recommendation module 340.

In some examples, the anatomy recognition, pathology recognition, and tool recognition modules 350 may also match billable templates (not shown) with any detected anatomies, pathologies, surgical tools, implants, sutures or the like included in the key frames from the key frame recommendation module 340. The billable templates, the key frames (e.g. the diagnostic key frames 341, the site preparation key frames 342, the suture passing key frames 343, the anchor insertion key frames 344, and/or the post treatment key frames 345 may be used to prepare the initial surgical report 360.

The initial surgical report 360 may also include text descriptions, in some cases based on the billable template information, that may be associated with key frames. In this manner, the initial surgical report 360 may include video images associated with the diagnostic key frames 341, the site preparation key frames 342, the suture passing key frames 343, the anchor insertion key frames 344, and/or the post treatment key frames 345 and descriptive text for the surgeon to review.

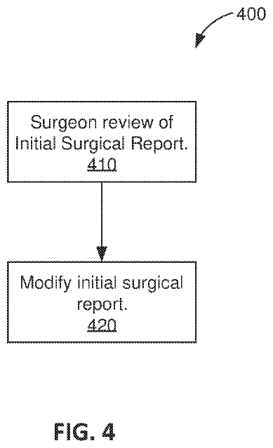

FIG. 4 is a flowchart depicting an example method 400 for modifying an initial surgical report. In block 410, an initial surgical report may be reviewed by the surgeon. In some examples, the initial surgical report may be the initial surgical report 280 of FIG. 2 or the initial surgical report 360 of FIG. 3.

The initial surgical report may be formatted for speedy review and approval by the surgeon. In some examples, the initial surgical report may include images, descriptive text, video clips of any surgical procedures performed by the surgeon. Upon review, the surgeon may note missing or erroneous information in the initial surgical report.

Next, in block 420 the surgeon modifies the initial surgical report. In some cases, the surgeon may add notes pertaining to the patient regarding the missing or erroneous information noted in block 410. In some cases, the surgeon may add information regarding information which are not visually perceptible, such as but not limited to, tissue quality, feel of the cartilage, and the like.

The modified initial surgical report may now be used to generate a surgery evidence report. The generation of the surgery evidence report is described in more detail below in conjunction with FIG. 5.

FIG. 5 shows an example schematic block diagram 500 for generating a billing report 580. The schematic block diagram 500 describes a relationship between various processing steps and surgical data that may be used to generate the billing report 580. In some examples, the processing steps may include executing neural networks with one or more processors (not shown).

First, surgical activity video segments (clips) 510 are obtained. The surgical activity video segments 510 may also include any video clips, and related text descriptions from the initial surgical report 280 of FIG. 2. In some examples, the surgical activity video segments 510 may also include video clips, annotations and corrections provided by the surgeon (for example as part of the modification of the initial surgical report in block 420 of FIG. 4). These annotations and corrections may reflect procedures that have taken place during the surgery, but were missing from the initial surgical report 280.

Next, the surgical activity video segments 510 may be processed by a tool recognizer module 520, an anatomy recognizer module 530, a surgical activity detector 540, and a pathology recognizer 550. The tool recognizer module 520, the anatomy recognizer module 530, the surgical activity detector 540, and the pathology recognizer 550 may be similar to the tool recognizer module 220, the anatomy recognizer module 230, the surgical activity detector module 243 and the pathology recognizer module 250 of FIG. 2. Therefore, in some variations the functionality of the tool recognizer module 520, the anatomy recognizer module 530, the surgical activity detector 540, and the pathology recognizer 550 may be substantially similar to the similarly named modules of FIG. 2. In some examples, the tool recognizer module 520, the anatomy recognizer module 530, the surgical activity detector 540, and the pathology recognizer 550 may include one or more neural networks that may be trained to provide any required functionality (similar functionality to as described for the similarly named modules of FIG. 2). Thus, the surgical activity detector 540 may detect surgical activity within the surgical activity video segments 510 based on data from the tool recognizer module 520, the anatomy recognizer module 530, and the pathology recognizer module 550.

The modifier detector 560 may receive data from the tool recognizer module 520, the pathology recognizer 550, and the surgical activity detector 540. In some examples, the modifier detector 560 may determine any modifications included in the surgical activity video segments 510. The modifications may be with respect to information included in the initial surgical report 280. For example, using recognized tool information (from the tool recognizer 520), identified pathologies and interoperative findings (from the pathology recognizer 550), and surgical video activity data (from the surgical activity detector 540), the modifier detector 560 may determine any modifications with respect to the surgical activity video segment 510. Thus, the modifier detector 560 may provide text descriptions, video clip information, and updated structured labels to identify any feasible updated or modified billable activities.

In some cases, the modifier detector 560 may retrieve report billable templates from the template database 570 and the template database 570. The report billable templates may provide descriptive text associated with approved billable surgical procedures associated with text descriptions, structured labels and/or video clip information. Thus the modifier detector 560 may provide updated entries to reflect modified or updated billable activities.

The billing report 580 may include video clips, key frames, text descriptions, and billable template data corresponding to the patient's surgical procedure. In this manner, all billable entries may include at least video clips or key frames to support the operative findings and surgical procedures performed on the patient.

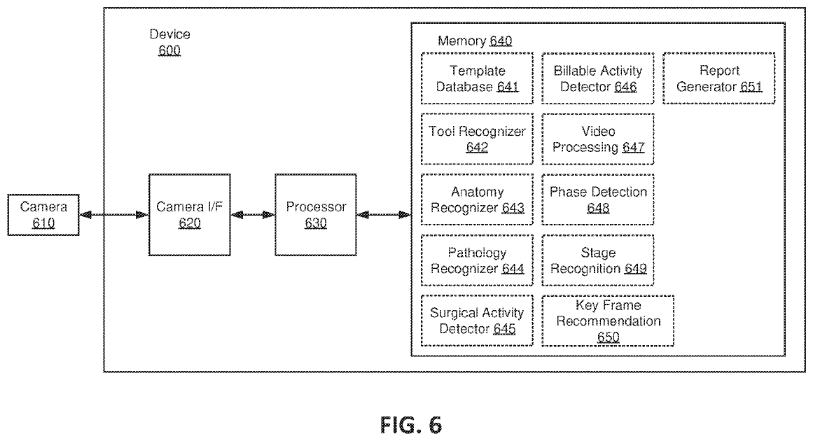

FIG. 6 shows a block diagram of a device 600 that may be one example of any device, system, or apparatus that may provide any of the functionality described herein. The device 600 may include a camera interface (I/F) 620, a processor 630, and a memory 640.

The camera I/F 620, which is coupled to the processor 630, may be used to interface with any feasible camera, such as a camera 610. In some examples, the camera 610 may be an orthoscopic camera.

The processor 630, which is also coupled to the memory 640, may be any one or more suitable processors capable of executing scripts or instructions of one or more software programs stored in the device 600 (such as within memory 640).

The memory 640 may include a template database 641 that may include a listing of structures (anatomies, implants, and the like) that may be necessary for a surgical procedure to be billable. The template database 641 may also include a text description of any feasible surgical procedure.

The memory 640 may also include a non-transitory computer-readable storage medium (e.g., one or more nonvolatile memory elements, such as EPROM, EEPROM, Flash memory, a hard drive, etc.) that may store the following software modules:
a tool recognizer module 642 to recognize and/or identify surgical tools;;
an anatomy recognizer module 643 to recognize and/or identify patient anatomy;
a pathology recognizer module 644 to recognize and/or identify pathologies;
a surgical activity detector module 645 to detect surgical activity in a surgical video;
a billable activity detector 646 to detect billable activities;
a video processing module 647;
a phase detection module 648 to detect surgical phases;
a stage recognition module 649 to recognize surgical stages;
a key frame recommendation module 650 to determine key video frames; and
a report generator module 651 to generate surgical or billing reports.
   Each software module includes program instructions that, when executed by the processor 630, may cause the device 600 to perform the corresponding function(s). Thus, the non-transitory computer-readable storage medium of memory 640 may include instructions for performing all or a portion of the operations described herein.

The processor 630 may execute the tool recognizer module 642 to identify and/or recognize surgical tools within video clips and/or video frames. In some examples, execution of the tool recognizer module 642 may cause the processor 630 to execute a neural network trained to identify and/or recognize any feasible surgical tools.

The processor 630 may execute the anatomy recognizer module 643 to identify and/or recognize patient anatomies within video clips and/or video frames. In some examples, execution of the anatomy recognizer module 643 may cause the processor 630 to execute a neural network trained to identify and/or recognize any feasible patient anatomy.

The processor 630 may execute the pathology recognizer module 644 to identify and/or recognize pathologies within video clips and/or video frames. In some examples, execution of the pathology recognizer module 644 may cause the processor 630 to execute a neural network trained to identify and/or recognize any feasible pathology.

The processor 630 may execute the surgical activity detector module 645 to identify and/or recognize surgical activity within video clips and/or video frames. In some examples, execution of the surgical activity detector module 645 may cause the processor 630 to execute a neural network trained to identify and/or recognize any feasible surgical activity.

The processor 630 may execute the billable activity detector module 646 to identify and/or recognize billable activities within video clips and/or video frames. In some examples, execution of the billable activity detector module 646 may cause the processor 630 to execute a neural network trained to identify and/or recognize any feasible billable activity.

The processor 630 may execute the video processing module 647 to identify and/or recognize video clips within one or more surgical videos. In some examples, execution of the video processing module 647 may cause the processor 630 to detect scene changes within surgical videos that may correspond to different video clips.

The processor 630 may execute the phase detection module 648 to identify and/or recognize different surgical phases. In some examples, execution of the phase detection module 648 may cause the processor 630 to detect or categorize one or more video clips as being associated with diagnostic, treatment, and/or post treatment surgical phases.

The processor 630 may execute the stage recognition module 649 to identify and/or recognize different surgical stages. In some examples, execution of the stage recognition module 649 may cause the processor 630 to detect or categorize one or more video clips as being associated with site preparation, suture passing, and/or anchor insertion surgical stages.

The processor 630 may execute the key frame recommendation module 650 to identify and/or recognize different key frames. In some examples, execution of the key frame recommendation module 650 may cause the processor 630 to determine whether any video frames are diagnostic, site preparation, suture passing, anchor insertion, and/or post treatment key frames.

The processor 630 may execute the report generator module 651 to generate initial surgical reports and/or billing reports.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A method of generating an initial surgical report describing detected billable activities, the method comprising:
receiving a surgical video of a surgical procedure performed on a patient;
identifying one or more surgical tools in the surgical video;
detecting surgical activity within the surgical video; and
determining one or more billable activities based on the identified surgical tools and the detected surgical activities.

2. The method of claim 1 further comprising:
recognizing a patient's anatomy in the surgical video, wherein determining the one or more billable activities is based, at least in part, on the recognized patient's anatomy.

3. The method of claim 2, wherein recognizing the patient's anatomy includes executing a neural network trained to recognize anatomy in a surgical video.

4. The method of claim 1, wherein identifying the one or more surgical tools includes executing a neural network trained to identify surgical tools in a surgical video.

5. The method of claim 1, further comprising:
recognizing a pathology in the surgical video, wherein determining the one or more billable activities is based, at least in part, on the recognized pathology.

6. The method of claim 5, wherein recognizing the pathology includes executing a neural network trained to recognize pathology in a surgical video.

7. The method of claim 1, wherein the billable activities include a video clip of the detected surgical activity.

8. The method of claim 1, wherein the billable activities include a descriptive text based at least in part on the detected surgical activity.

9. The method of claim 1, further comprising:
generating an initial surgical report based at least in part on the one or more determined billable activities.

10. The method of claim 1, wherein the surgical video is captured with an orthoscopic camera.

11. The method of claim 1, wherein detecting surgical activity includes executing a neural network trained to detect surgical activity in a surgical video.

12. A system comprising:
one or more processors; and
a memory configured to store instructions that, when executed by the one or more processors, cause the system to:
receive a surgical video of a surgical procedure performed on a patient;
identify one or more surgical tools in the surgical video;
detect surgical activity within the surgical video; and
determine one or more billable activities based on the identified surgical tools and the detected surgical activities.

13. A method of providing an initial surgical report describing detected billable activities, the method comprising:
determining a plurality of video clips from a surgical video of a surgical procedure performed on a patient;
determining a plurality of recommended key frames from the plurality of video clips; and
determining one or more billable activities based on the plurality of key frames.

14. The method of claim 13, further comprising:
detecting a plurality of surgical phases from the plurality of video clips, wherein the key frames are based, at least in part, on the plurality of surgical phases.

15. The method of claim 14, further comprising:
recognizing one or more stages within at least one of the plurality of surgical phases, wherein the key frames are based, at least in part, on the one or more stages.

16. The method of claim 13, wherein the key frames include diagnostic key frames, site preparation key frames, suture passing key frames, anchor insertion key frames, post treatment key frames, or a combination thereof.

17. The method of claim 13, further comprising:
generating an initial surgical report based at least in part on the key frames.

18. The method of claim 13 further comprising recognizing patient anatomy in one or more of the key frames, wherein the billable activities are based, at least in part, on the recognized patient anatomy.

19. The method of claim 18, wherein recognizing patient anatomy includes executing a neural network trained to recognize patient anatomy.

20. The method of claim 13, further comprising recognizing a pathology in one or more of the key frames, wherein the billable activities are based, at least in part, on the recognized pathology.

21. The method of claim 20, wherein recognizing the pathology includes executing a neural network trained to recognize patient pathology.

22. The method of claim 13, further comprising recognizing a surgical tool in one or more of the key frames, wherein the billable activities are based, at least in part, on the recognized surgical tool.

23. The method of claim 22, wherein recognizing the surgical tool includes executing a neural network trained to recognize one or more surgical tools.

24. A system comprising:
one or more processors: and
a memory configured to store instructions that, when executed by the one or more processors, cause the system to:
determine a plurality of video clips from a surgical video of a surgical procedure performed on a patient;
determine a plurality of recommended key frames from the plurality of video clips; and
determine one or more billable activities based on the plurality of key frames.

25.-30. (canceled)
